(19) United States
(12) Patent Application Publication
(10) Pub. No.: US 2010/0305595 A1
(43) Pub. Date: Dec. 2, 2010

(54) Surgical instrument

(75) Inventor: Reiner Hermann, Fridingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/800,050

(22) Filed: May 5, 2010

(30) Foreign Application Priority Data
May 29, 2009 (DE) 10 2009 024 244
Aug. 5, 2009 (DE) 10 2009 037 153

(51) Int. Cl. A61B 17/32

(52) U.S. Cl. 606/170

(57) ABSTRACT

A surgical instrument is suggested with a proximal and a distal end section, comprising an elongated, hollow outer shaft which extends from the proximal to the distal end section, a hollow cylindrical drive element rotatably mounted in the outer shaft as well as a cutting, abrading or milling tool which is arranged at the distal end section of the instrument and coupled to the drive element, wherein the drive element has a flexible section which is arranged between the proximal and distal end sections and comprises a plurality of ring segments which each have, in an axial direction, a first and a second end region, wherein the first end region comprises two or more projections protruding in an axial direction and the second end region comprises two or more recesses accommodating the projections and the ring segments engage in one another in a joint-like manner via the projections and recesses, characterized in that the ring segments are connected to one another in a form locking manner in an axial and/or radial direction via the projections and recesses.

The surgical instrument according to the invention makes a reliable operation possible, in particular at high rotational speeds as well, with minimal resources for the production.

This application claims the benefit of German Patent Application No. 10 2009 024 244.9 filed on May 29, 2009 and German Patent Application No. 10 2009 037 153.2 filed on Aug. 5, 2009.

The present disclosure relates to the subject matter disclosed in German applications number 10 2009 024 244.9 of May 29, 2009 and number 10 2009 037 153.2 of Aug. 5, 2009, which are incorporated herein by reference in their entirety and for all purposes.

### BACKGROUND OF THE INVENTION

The invention relates to a surgical instrument for minimally invasive surgical procedures with a tool coupled to a drive element at a so-called distal end of a shaft. Such instruments are also often called shavers.

Surgical instruments of this type have a proximal and a distal end section, an elongated, hollow outer shaft, a hollow cylindrical drive element which is rotatably mounted in the outer shaft as well as a cutting, abrading or milling tool which is arranged at the distal end section of the instrument and coupled to the drive element.

Apart from instruments which are designed in a straight line, surgical instruments are also known, with which the distal end region is angled or curved in order to be able to also reach operating positions which are more difficult to access with the surgical instrument and in order to generally increase the size of the operating area of the instrument.

It is known in this connection to equip the drive element between the proximal and distal end sections with a flexible section which comprises a plurality of ring segments which each have a first and a second end region in axial direction, wherein the first end region comprises two or more projections protruding in an axial direction and the second end region comprises two or more recesses accommodating the projections and the ring segments engage in one another in a joint-like manner via the projections and recesses.

Surgical instruments of this type are known, for example, from EP 0 677 276 B1, wherein the flexible section allows the torque to be transferred effectively from the proximal end section of the drive element to the distal end section and, therefore, to the tool connected thereto.

Since the shaver typically serves the purpose of removing body tissue, the channel present in the interior of the shaft and the drive element is used for removing the separated tissue parts by suction.

The disadvantage of these known instruments is that the production, in particular the assembly, of the flexible section is complicated since the flexible section is only loosely put together from a plurality of ring segments and so ring segments can be lost not only during the production but also during any later disassembly of the drive element and its removal from the hollow cylindrical, outer shaft, also, in particular, during an operation.

A further shaver, which follows this principle of the construction of the drive element with ring segments engaging loosely in one another in the flexible section, is known from DE 10 2004 046 539 A1.

Another starting point has been selected in EP 0 986 989 B1, in which the flexible section is formed by a hollow cylindrical element, the wall of which is slit in a radial direction in the form of a helical line, wherein teeth and indentations alternate along the helical line in a meandering, alternating manner and engage in one another so that the windings are held together in an axial direction.

The disadvantage of this solution is a considerably reduced flexibility and fatigue fractures which often occur as a result of the alternating bending stress during the rotation of the shaft. An oscillating drive is intended to be possible with this instrument but it can be driven with high rotational speeds only in one direction of rotation.

The object of the invention is to further develop a surgical instrument of the type described at the outset such that a reliable operation, in particular at high rotational speeds as well, is possible with minimal efforts for the production.

### SUMMARY OF THE INVENTION

This object is accomplished in accordance with the invention, in a surgical instrument, in that the ring segments are connected to one another in a form locking manner (also known as positive locking or form fit) in an axial and/or radial direction via the projections and recesses.

The handling of the drive element is made considerably easier during the production, the assembly as well as during any later disassembly as a result of the form locking connection of the projections and recesses in an axial and/or radial direction, preferably in an axial and radial direction. The joint-like connection of the ring segments in the flexible section is, nevertheless, ensured.

If the ring segments are provided with more than two projections and recesses, several joint axes may be realized per pair of ring segments.

The form locking connection in an axial or radial direction reduces the risk of individual ring segments becoming lost to a considerable extent when their loss is not precluded from the start by the preferably axial and radial form locking connection.

The reciprocal positioning of the ring segments bordering on one another can also be predetermined via the form locking connection in an axial and/or radial direction so exactly that high rotational speeds can also be realized in both possible drive directions.

The form locking connection in an axial direction may be improved by the use of more than two projections and recesses.

The projections and recesses will typically be arranged on the respective ring segment at regular intervals in a circumferential direction. This ensures an even load on the ring segments during the transfer of the drive forces and, therefore, as long a service life as possible.

According to one embodiment of the present invention, the number of projections and recesses is selected to be uneven, which results in a form locking connection in a radial direction without additional measures, in particular in the case of an arrangement of the projections and recesses on the respective ring segment at regular intervals in a circumferential direction. In addition or alternatively, the form locking connection can be achieved in a radial direction in that in circumferential direction the projections have a greater extension on their side located radially outwards than on their side located radially inwards and that in circumferential direction the recesses have, accordingly, an extension on their side located radially inwards which is smaller than the corresponding extension located radially outwards in the corresponding region of the projection engaging in the recess.

A form locking connection in a radial direction can be achieved with this measure, irrespective of whether the number of projections and recesses is uneven or even.

The form locking connection in an axial direction can be achieved in that in circumferential direction the projections have a greater extension at their free end facing away from the ring segment than at their end adjacent to the ring segment and in circumferential direction the recesses have a smaller extension at their open end than the extension of the projection, which engages in the recess, at its free end.

If a form locking connection is realized not only in an axial direction but also in a radial direction, a drive element is present which can be handled as a whole without the risk of individual parts of the flexible section becoming lost.

Such a drive element is preferably produced with a flexible section such that, first of all, a one-piece, hollow cylindrical element is used, into which the contours of the projections and recesses are cut, for example, with a laser. Often, the cut gap resulting thereby is sufficient for ensuring an adequate pivoting of the individual ring segments relative to one another and so the flexible section of the drive element can provide for an adequate bending angle for the respective application.

If a form locking connection is already present in an axial direction, the form locking connection in a radial direction can also be achieved in that the projections have, in axial direction, a greater extension in a longitudinal direction on the side located radially outwards than on the side located radially inwards and the recesses have, in axial direction, a depth on the side located radially inwards which is smaller than the length of the projections in an axial direction on their side located outwards.

In one preferred embodiment of the invention, contact surfaces which are flat are provided in circumferential direction on sides of the projections as well as correspondingly in the case of the recesses. As a result, the transfer of torque is optimized such that less wear and tear and the possibility of allowing very high rotational speeds result.

One preferred shape of the projections in circumferential direction is the trapezoidal shape even if, of course, shapes deviating herefrom make the realization of a form locking connection possible. Corresponding thereto, the recesses are preferably of a trapezoidal design in circumferential direction.

This results in large, flat contact surfaces of the projections and recesses.

In order to guarantee a form locking connection in a radial direction, the projections are preferably designed with a trapezoidal cross section when seen in the radial direction, wherein the curvatures on the surfaces of the projections which are located radially outwards and radially inwards are not taken into consideration in this definition.

Corresponding hereto, the recesses likewise preferably have a trapezoidal cross section in a radial direction.

These and other advantages of the invention will be explained in greater detail in the following on the basis of the drawings.

### BRIEF DESCRIPTION OF THE DRAWINGS

FIG. 1 shows a perspective illustration of a surgical instrument according to the invention in the form of a shaver;

FIG. 2 shows an enlarged section of the shaver from FIG. 1;

FIG. 3 shows an alternative embodiment of the distal end of FIG. 2;

FIG. 4 shows a detail of the flexible drive element;

FIG. 5 shows an alternative embodiment of part of the flexible drive element; and

FIG. 6 shows a schematic illustration of the type of use of the surgical instrument according to the invention.

### DETAILED DESCRIPTION OF THE INVENTION

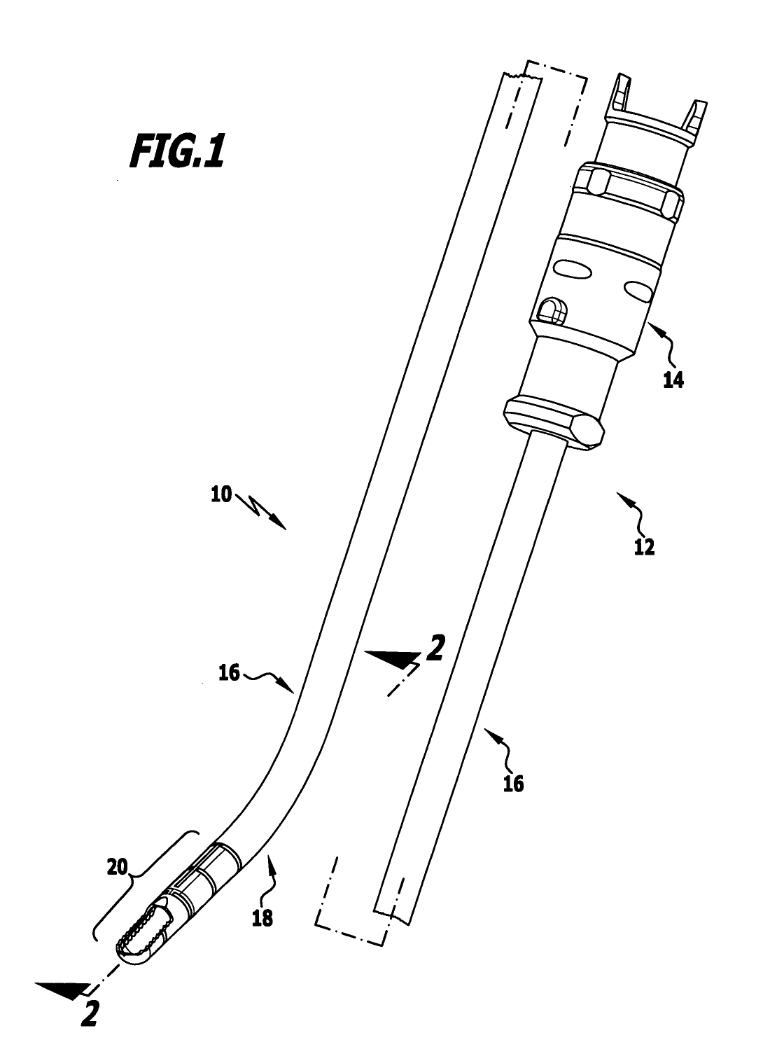

FIG. 1 shows a surgical instrument according to the present invention which is designated altogether with the reference numeral 10 and will also, in the following, be called a shaver for short.

The shaver 10 according to the invention has at its proximal end 12 a coupling device 14 which can be connected to a surgical drive (not illustrated).

On the distal side, the coupling device 14 is adjoined by a shaft 16 which is resistant to bending and torsion, is inclined in the region of its distal end section 18 through approximately 20° in comparison with the longitudinal axis of the shaver 10 and comprises a distal shaft section 20.

The coupling device 14 is already shown and described in detail in DE 10 2004 046 539 A1 and so reference can be made to this description with respect to the details.

The shaft 16 with its distal end section 20 is designed as an elongated outer sleeve 22, in which a hollow cylindrical drive element 24 is guided. The shaft 16 or rather the sleeve 22 is closed at the distal end and provided with a lateral opening 26 which creates a passage from the interior space of the sleeve 22 to the surroundings of the shaver 10.

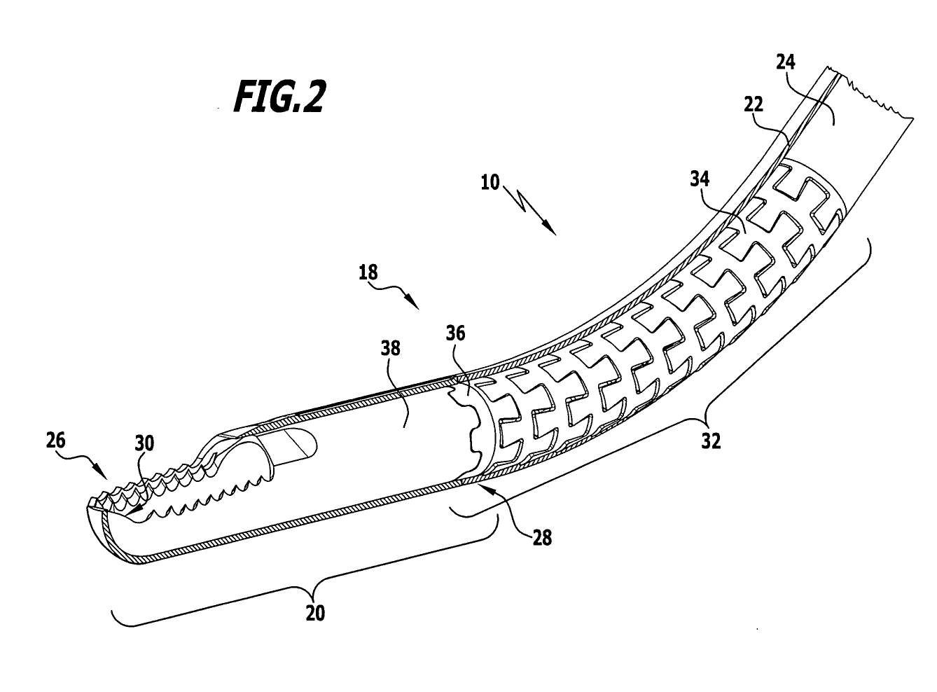

A tool 38 is coupled to the drive element 24 at its distal end 28 and this tool likewise has a lateral opening 30 which is designed to essentially correspond to the lateral opening 26 of the sleeve 22. The openings 26 and 30 are, as is apparent in FIG. 2, ground at their edges and provided with cutting edges. If the drive element 24 is caused to rotate, tissue parts reaching into the area of the lateral openings 26, 30 will be separated by the cutting edges of the edges of the openings 26, 30 and can be drawn off by means of an underpressure via the interior space remaining in the drive element 24.

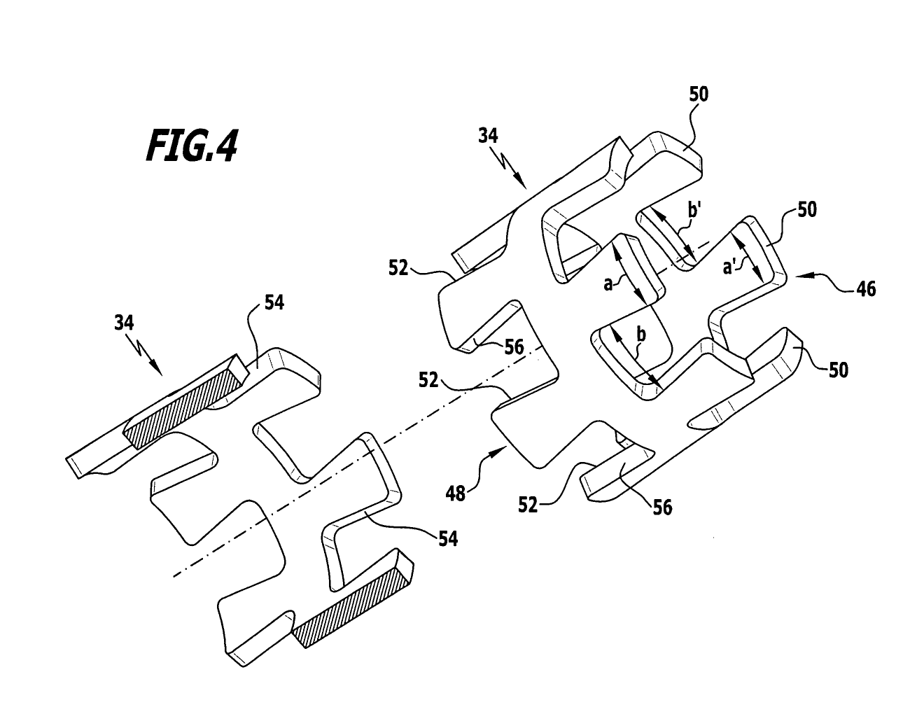

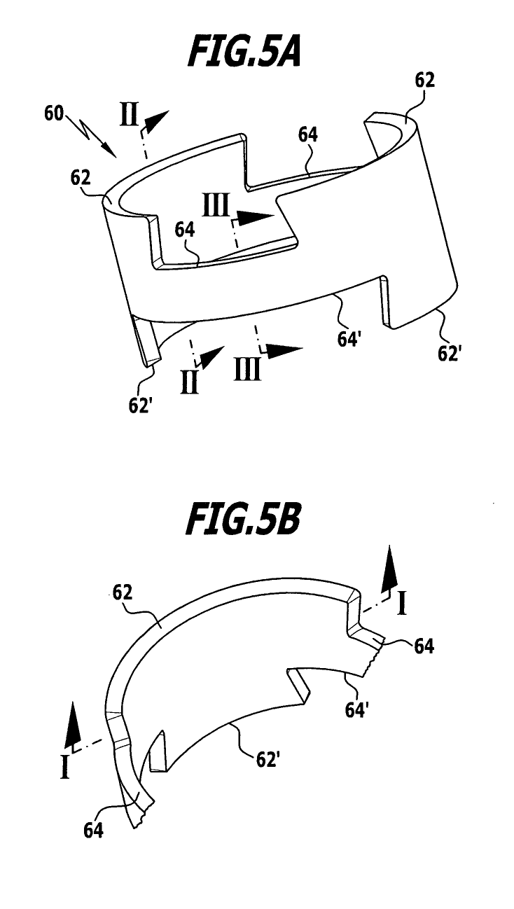

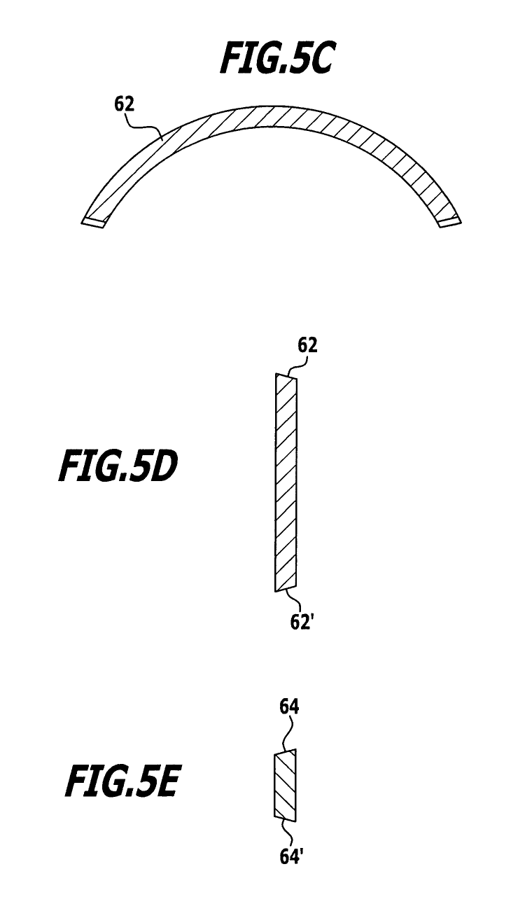

In the end section 18 of the shaver 10, the hollow cylindrical drive element 24 merges from a one-piece or solid hollow cylindrical part into a hollow cylindrical, flexible section 32 which is formed by a number of ring segments 34 which will be described in greater detail in the following on the basis of FIGS. 4 and 5.

The distal end of the drive element 24 terminates with a coupling piece 36, to which a tool, in particular a milling tool 38, can be coupled in a loosely engaging manner.

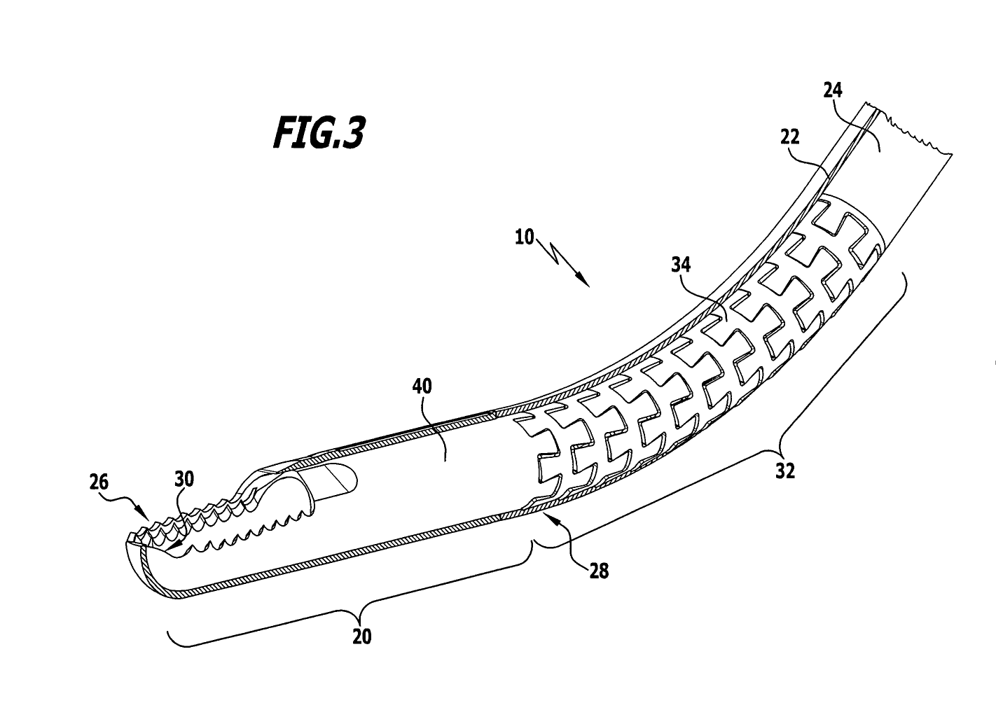

FIG. 3 shows, on the other hand, the distal end 20 of the shaver 10 in an alternative embodiment, with which, instead of the tool 38 coupled via a coupling piece 36, a tool 40 is held undetachably in a form locking connection.

Differences result in the case of these two embodiments in the manufacture of the instruments:

In the embodiment of FIG. 2, the tool 38 is introduced into the sleeve 22 first of all during assembly, wherein the distal end section is then bent and brought into its final shape. After that, the drive element 24 may be inserted into the sleeve with its flexible section 32, wherein the coupling piece 36 engages in the proximal end of the tool 38.

In the embodiment of FIG. 3, on the other hand, the drive element 24 is manufactured together with the tool 40 and pushed into the sleeve 22 as long as this still has a straight shape. Only thereafter will the sleeve 22 be bent and brought into its final, curved shape.

The ring segments 34 are illustrated in detail in FIG. 4 and have a first end region 46 in a first axial direction and a second end region 48 in the opposite axial direction. At its first end region, the ring segment 34 has six projections 50 which are arranged so as to be distributed on the ring segment 34 in a regular manner in circumferential direction. At its axially opposite end region 48, the ring segment 34 has recesses or setbacks 52 shaped to correspond to the projections 50, wherein the projections and recesses each have a trapezoidal shape when seen in circumferential direction, wherein in the case of the projections 50 the free end of the projections 50 in an axial direction has a greater extension in circumferential direction than the end located adjacent to the ring segment while the recesses 52 have, accordingly, at their inner end a greater extension in circumferential direction than the outwardly open sections located in the direction towards the end region 48. In the case of the ring segments 34, which are shown in detail in FIG. 4, both end regions are provided with a configuration which is identical except for the mirror-image arrangement on account of the regular geometry.

As a result of the fact that projections 50 engage in correspondingly shaped recesses 52 with clearance and, nevertheless, in a positive or form locking manner, an axial form locking connection results and so the ring segments 34 can be connected to one another in an axial direction and thus be introduced together into the outer sleeve 22 or be withdrawn from it.

Nevertheless, sufficient clearance remains on account of the existing gap between the projections 50 and the recesses 52 to ensure a joint-like connection of adjacent ring segments 34.

The projections 50 and the recesses 52 each have contact surfaces 54, 56 which come into contact with one another and are designed to be flat and of a large surface area in the embodiment shown in FIG. 5. As a result, an optimum transfer of torque between the individual ring segments which follow one another is possible.

On account of the plurality of projections and recesses 50, 52, several pivot axes result for the connection of two consecutive ring segments and so the drive element 24 can follow the curved course of the hollow cylindrical shaft 16 exactly.

On account of the plurality of projections and recesses 50, 52 with a trapezoidal configuration which engage in one another, a particularly secure connection results in an axial direction.

In order to be able to handle the drive element 24 with its flexible section 32 and the ring segments 34 forming it in an altogether problem-free manner, it is provided for the extension of the projections 50 at their free end to be greater in circumferential direction (a) than their corresponding extension on the inner side (a′).

Accordingly, the recesses 52 are dimensioned at their outer and inner circumference with a width b and a width b′, respectively, which also make a form locking connection possible in a radial direction and so the ring segments 34 are undetachably connected to one another and can be handled as such.

Whereas in the case of the ring segments of FIG. 4, six projections and six recesses are respectively provided, in an alternative embodiment of the ring segments 60 in FIG. 5, which illustrate the simplest embodiment, only two projections 62 and two recesses 64 are respectively present at the axially opposite ends of the ring segment 60 but they can be used in a similar manner for the realization of the present invention. Only the realizable pivot angle of two ring segments 60 bordering on one another is restricted somewhat in comparison with two ring segments 34 which border on one another.

In this case, as well, the projections and recesses 62, 64 are intended to be connected to one another in a form locking manner in an axial and/or radial direction and so reference can be made to the explanations concerning FIG. 4 with respect to the configuration of the projections 50 and the recesses 52, respectively.

The sectional illustrations of FIGS. 5b to 5e clarify the trapezoidal cross sections of the projections 62 and the recesses 64 in circumferential as well as in axial direction. This also applies for the design of the projections and recesses of the ring segments 34.

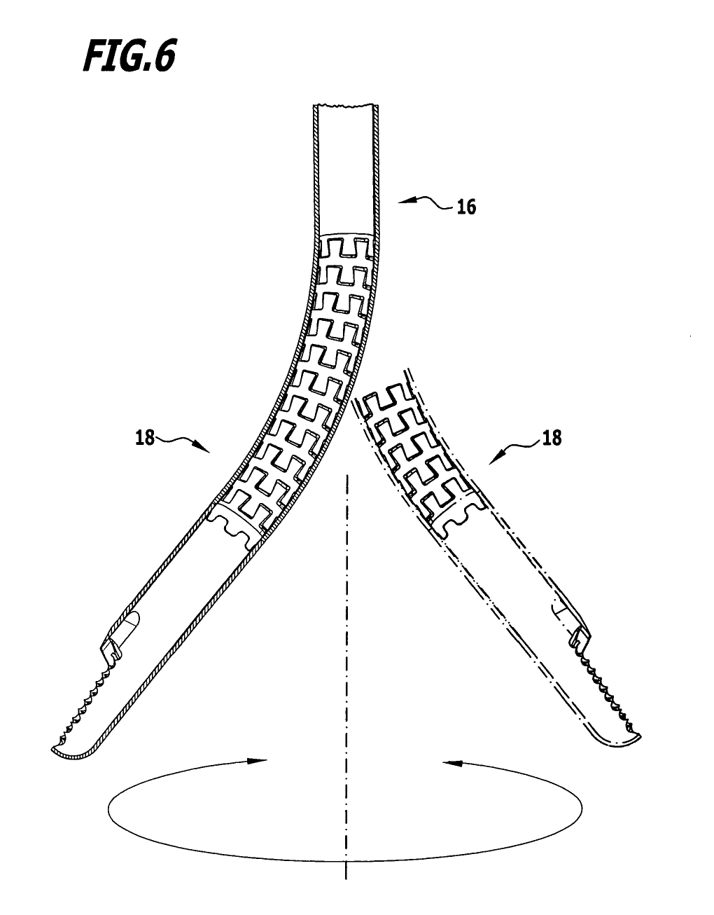

Finally, the overall view in FIG. 6 indicates what significance the angled region in the distal end section 18 of the shaft 16 has for the operating area which can be reached with the shaver and which is considerably larger as a result of the curvature illustrated in FIG. 6 than in the case of a shaft which is designed only in a straight line.

1. Surgical instrument with a proximal and a distal end section, comprising an elongated, hollow outer shaft extending from the proximal to the distal end section, a hollow cylindrical drive element rotatably mounted in the outer shaft as well as a cutting, abrading or milling tool arranged at the distal end section of the instrument and coupled to the drive element,
wherein the drive element has a flexible section arranged between the proximal and distal end sections and comprising a plurality of ring segments each having in an axial direction a first and a second end region, wherein the first end region comprises two or more projections protruding in an axial direction and the second end region comprises two or more recesses accommodating the projections and the ring segments engage in one another in a joint-like manner via the projections and recesses, wherein the ring segments are connected to one another in a form locking manner in an axial and/or radial direction via the projections and recesses.

2. Instrument as defined in claim 1, wherein the projections and recesses are arranged on the respective ring segment at regular intervals in a circumferential direction.

3. Instrument as defined in claim 1, wherein the number of projections and recesses is uneven.

4. Instrument as defined in claim 1, wherein in circumferential direction the projections have a greater extension on their side located radially outwards than on their side located radially inwards and wherein in circumferential direction the recesses have an extension on their side located radially inwards smaller than the corresponding extension located radially outwards of the projection engaging in the recess.

5. Instrument as defined in claim 1, wherein in circumferential direction the projections have a greater extension at their free end facing away from the ring segment than adjacent to the ring segment and wherein in circumferential direction the recesses have a smaller extension at their open end than the extension of the projection engaging in the recess at its free end.

6. Instrument as defined in claim 1, wherein in axial direction the projections have a greater extension on the side located radially outwards than on the side located radially inwards and wherein in axial direction the recesses have a depth on the side located radially inwards smaller than the length of the projections in axial direction on their side located outwards.

7. Instrument as defined in claim 1, wherein the projections are of a trapezoidal design in circumferential direction.

8. Instrument as defined in claim 1, wherein the recesses are of a trapezoidal design in circumferential direction.

9. Instrument as defined in claim 1, wherein the projections have a trapezoidal cross section in a radial direction.

10. Instrument as defined in claim 1, wherein the recesses have a trapezoidal cross section in a radial direction.

11. Instrument as defined in claim 1, wherein the projections and recesses have flat contact surfaces coming into contact with one another in circumferential direction.
